(19) United States
(12) Patent Application Publication
(10) Pub. No.: US 2011/0250302 A1
(43) Pub. Date: Oct. 13, 2011

(54) Composition for Preventing Hair Loss and Promoting Hair Regrowth

(76) Inventor: In-Sun Ko, Jeju-si (KR)

(21) Appl. No.: 13/031,140

(22) Filed: Feb. 18, 2011

(30) Foreign Application Priority Data
Apr. 7, 2010 (KR) 10-2010-0031760
Oct. 15, 2010 (KR) PCT/KR2010/007091

(51) Int. Cl. A61K 36/73; A61P 17/14

(52) U.S. Cl. 424/765

(57) ABSTRACT

Disclosed is a composition for preventing hair loss and promoting hair regrowth. It acts to inhibit the activity of 5α-reductase and is useful as a hair regrowth agent as assayed in animal and clinical trials.

### CROSS-REFERENCE TO RELATED APPLICATIONS

This application claims priority to Korean Application no. 10-2010-0031760, filed on Apr. 7, 2010, and claims priority to International Application no. PCT/KR2010/007091, filed on Oct. 15, 2010; both of which are hereby incorporated by reference for all purposes.

### BACKGROUND OF THE INVENTION

1. Field of the Invention

The present invention relates to a composition for preventing hair loss and promoting hair regrowth. More particularly, the present invention relates to a composition for preventing hair loss and promoting hair growth using an Indian strawberry (Duchesnea chrysantha) extract.

2. Description of the Related Art

In recent years, an increasing number of people have suffered from hair loss due to various factors including environmental pollution, side effects of drugs, social-environmental stress, and the like.

Hair follicle growth occurs in cycles, each consisting of a long growing phase (anagen), a short transitional phase (catagen) and a short resting phase (telogen). In persons with hair loss, the number of hair in anagen phase decreases while the number in catagen and telogen phases increases.

Causes of hair loss, although not yet clear thus far, are known to include poor blood circulation, excessive sebum secretion induced by dihydrotestosterone, peroxide or bacteria-induced scalp disorders, aging, genetic factors, stress, and combinations thereof.

High levels of dihydrotestosterone have been discovered in patients with hair loss leading and this androgen is now regarded as a main cause of hair loss.

Dihydrotestosterone is an androgen synthesized from the male testosterone by the action of 5 α-reductase.

A drug which suppresses the activity of 5 α-reductase to block the synthesis of dihydrostestosterone was developed as a therapeutic treatment for hair loss. For example, one of the drugs approved by the FDA's is Propecia (finasteride), manufactured by Merck, U.S. (U.S. Pat. Nos. 5,547,957 and 5,760,046 or Korean Patent No. 375083).

The Indian strawberry extract according to the present invention was also found to suppress the activity of 5 α-reductase and prevent hair loss and promote hair regrowth as measured in animal and clinical trials.

### SUMMARY OF THE INVENTION

Accordingly, the present invention has been made keeping in mind the above problems occurring in the prior art, and an objective of the present invention is to provide a composition for preventing hair loss and promoting hair regrowth. Other objectives and aspects of the present invention are disclosed below.

### DESCRIPTION OF THE PREFERRED EMBODIMENTS

As will be illustrated in detail in the following examples, extracts obtained from combining dried Indian strawberry herba with water and 70% ethanol were assayed for inhibitory activity against 5 α-reductase, an enzyme involved in the production of dihydrotesterone responsible for hair loss. When compared to a control (treated with no Indian strawberry extracts), the extracts were observed to increase the inhibitory activity water by 64.3% (water extracts) and 58.7% (ethanol extracts). Similar results were obtained in both animal and clinical trials.

Notably, when used in combination with a red ginseng extract or a Zanthoxylum planispinum leaf extract, the Indian strawberry extracts of the present invention show higher inhibitory activity against 5α-reductase, and their excellent hair regrowth promoting and/or hair loss-preventing property was also detected in animal and clinical trials.

For example, a mixture of the Indian strawberry extract and the red ginseng extract at a ratio of from 9:1 to 7:3 (weight ratio) was superior to the Indian strawberry extract alone in terms of the activities of both inbibiting 5 α-reductase and promoting hair regrowth. At a ratio of 8:2 the Indian strawberry extract and the ginseng extract showed higher effects than did the commercially available hair regrowth product Moxidil® (Hammi Pharmaceutical, Korea).

Also, a ratio of 9:1 (weight) Indian strawberry extract and Zanthoxylum planispinum leaf extract shows higher desired effects than does Moxidil® (Hanmi Pharmaceutical, Korea).

The present invention is provided based on experimental results.

The composition for preventing hair loss and promoting hair regrowth according to the present invention is characterized in that it comprises an Indian strawberry extract as an active ingredient, preferably in combination with a red ginseng extract/powder or a soapberry leaf extract.

The term “hair loss,” as used herein, is intended to mean all symptoms accepted as “hair loss” in the art irrespective of direct and indirect causes. The symptoms of hair loss caused by poor blood circulation, excessive sebum secretion induced by dihydrotestosterone, peroxide or bacteria-induced scalp disorders, aging, genetic factors, stress, and combinations thereof fall into the scope of the present invention. This is as shown in the following Experimental Examples (Experimental Example 3). In this experiment, 150 randomly selected men suffering from hair loss were treated with or without the Indian strawberry extracts of the present invention, and the effect of preventing hair loss or promoting hair regrowth was detected in all the three groups of 30 men who were treated with the Indian strawberry extract.

As used herein, the “Indian strawberry extract” is intended to include crude extracts obtained when Indian strawberry is immersed in water, e.g., distilled water, lower alcohol products of 1-4 carbon atoms, such as methanol, ethanol, butanol, etc., or a combination thereof, regardless extraction methods, and purified extracts obtained by fractioning the crude extracts with the solvents. Concentrated extracts in a liquid or solid form from which the solvent is removed are also within the scope of the present invention. Provided it uses an immersion method, any extraction method may be used in the present invention. Examples of the extraction methods useful in the present invention include cold-precipitation, refluxing, heating, and microwaving. It is preferable that an Indian strawberry extract is obtained using water, ethanol or a combination thereof.

The “red ginseng extract” or the “Zanthoxylum planispinum leaf extract” herein can be as defined in the “Indian strawberry extract” with an exception that the subject to be extracted is red ginseng or zanthoxylum planispinum.

The term “red ginseng” as used herein means red ginseng obtained by steaming raw ginseng and drying it. More particularly, it is red ginseng obtained by steaming raw ginseng and drying it to a water content of 10˜15 wt % in a sunny or shady place.

Even though a red ginseng extract was used, it is obvious to those skilled in the art that a red ginseng powder shows similar effects. Hence, a red ginseng powder may be used instead of or in combination with a red ginseng extract.

As stated above, when the Indian strawberry extract is used in combination with the red ginseng extract, an increased effect of promoting hair regrowth and preventing hair loss was obtained. Particularly when the Indian strawberry extract is mixed at a weight ratio of 9:1 to 7:3 with a red ginseng extract, the best effects were obtained. Therefore, the Indian strawberry extract is preferably mixed at a weight ratio of 9:1 to 7:3 with the red ginseng extract. The weight ratio is based on the condition that the extracts are in the same form when they are mixed. For example, if the Indian strawberry extract is in a solid form, the ratio is based on the state where the red ginseng extract takes a solid form free of solvents. Likewise, if the Indian strawberry extract is in a liquid form, the red ginseng extract should also be in a liquid form.

The “active ingredient” herein means an ingredient which shows the desired effect when it used solely or along with a carrier having no effect. Unless otherwise defined, terms used herein accept dictionary based definitions or meanings generally accepted in the related art.

In the composition for preventing hair loss and promoting hair regrowth according to the present invention, the amount of Indian strawberry extract may vary depending on various factors including uses, dosage forms, formulations, etc. as long as the composition guarantees the effect of preventing hair loss and promoting hair regrowth. An effective amount of the Indian strawberry extract may be within the range of from 0.001% to 99.999% by weight based on the total weight of the composition. The “effective amount” as used herein means an amount of the active ingredient which is effective in preventing hair loss and promoting hair regrowth. Such an effective amount can be experimentally determined within the typical ability of those skilled in the art.

In addition to the Indian strawberry extract, the composition for preventing hair loss and promoting hair regrowth in accordance with the present invention may comprise other compounds or plant extracts which are known to have the effect of preventing hair loss and promoting hair regrowth for a synergistic increase. Further, the composition may comprise a compound or plant extract used in preparing a hair tonic.

Examples of the compound or plant extract include a female hormone such as progesterone or estrogen, a green tea extract, finasteride, a biota seed extract, a sophora flavescens aiton extract, linoleic acid, gamma linoleic acid, jojoba oil, olive oil, sunflower oil, avocado oil, soybean oil added lecithin, a clove extract, a coix extract, a gallnut extract, a curcuma longa extract, a areca nut extract, a catechu extract, a pharbitis extract, a plantage seed extract, a lignum acronychiae extract, a licorice extract, a psoraleae fructus extract, saw palmetto, an apple extract, a swertia pseudochinensis extract, a ginseng extract, a rehmannia glutinosa extract, a pleuropterus multiflorus extract, a polygonati rhizoma extract, pantothenyl ethyl ether, D-panthenol, a henna extract, an iris extract, hinokitiol, nicotinic acid amide, L-menthol, minoxidil, a ginkgo extract, tocopherol acetate, nicotinic acid benzyl, salicylic acid, piroctone olamine, and ketoconazole.

One or more of the exemplary compounds and plant extracts may be used together with the Indian strawberry extract in the composition for preventing hair loss and promoting hair regrowth according to the present invention.

In an embodiment, the composition for preventing hair loss and promoting hair regrowth in accordance with the present invention is provided as a hair tonic.

When the composition for preventing hair loss and promoting hair regrowth of the present invention is provided as a hair tonic, it can be prepared in various formulations such as emulsions, lotion, creams (oil in water type, water in oil type, multiphase), solutions, suspensions (anhydrous and hydrous), anhydrous products (oily and glycol), gels, powders, etc.

The hair tonic composition for preventing hair loss and promoting hair regrowth according to the present invention may comprise a cosmetically acceptable carrier in addition to the Indian strawberry extract.

Examples of the carrier include alcohols, oils, surfactants, fatty acids, silicone oils, antiseptics, wetting agents, humectants, viscosity modifiers, emulsifiers, stabilizer, sunscreen agents, colorants, perfume, and diluents.

Compounds or compositions which may be used as alcohols, oils, surfactants, fatty acids, silicone oils, antiseptics, wetting agents, humectants, viscosity modifiers, emulsifiers, stabilizer, sunscreen agents, colorants, perfume, and diluents are known in the art and may be readily selected by those skilled in the art.

Among the alcohols are higher alcohols and water-soluble polyhydric alcohols including propylene glycol, 1,3-butylene glycol, glycerin, sorbitol and polyethylene glycol. Avocado oil, palm oil, beef tallow, and jojoba oil are suitable for use. Ethyl paraben and butyl paraben fall within the scope of the antiseptics. Hyaluronic acid, chondroitin sulfate and pyrrolidone carboxylate may be used as moisturizing agents. The diluents may be exemplified by ethanol and isopropanol.

In another embodiment of the present invention, the composition for preventing hair loss and promoting hair regrowth according to the present invention is provided as a shampoo composition.

When prepared in a shampoo composition, the composition of the present invention includes a carrier suitable for use in shampoo compositions in addition to the Indian strawberry extract. Examples of the carrier include surfactants, foam enhancers, emulsifiers, thickeners, hydrotropes, antiseptics, ph modifiers, germicides, anti-dandruff agents, tonics, and moisturizing agents.

Surfactants, foam enhancers, emulsifiers, thickeners, hydrotropes, antiseptics, ph modifiers, germicides, anti-dandruff agents, tonics, and moisturizing agents are known in the art. For example, alkyl ether sulfates (alcohol ethoxy sulfate, sodium lauryl ethoxylated sulfate), alkyl sulfates (alcohol sulfate), acylsarcosinate, acylglutaminate, phosphate ester, sucrose ester may be used as surfactants. Foam enhancers may be exemplified by alkanolamide and amine oxide. Among the thickeners are sodium chloride, sodium sulfate, starch, and dextrin. Ethyl alcohol, butyl alcohol, propylene glycol are known as hydrotropes. Glycerin serves as a moisturizing agent.

In still another embodiment of the present invention, the composition according to the present invention is provided as a soap composition.

When prepared in a soap composition, the composition of the present invention may comprise a soap base and an additive used in soap, such as skin humectants, emulsifiers and hard water softeners, in addition to the Indian strawberry extract.

Soap is based on a vegetable oil, such as coconut oil, palm oil, soybean oil, castor oil, olive oil or palm kernel oil, or an animal fat, such as beef tallow, lard, brisket and flank, or fish oil. Examples of the skin humectants include glycerin, erythritol, polyethylene glycol, propylene glycol, butylene glycol, pentylene glycol, hexyl glycol, isopropyl myristate, silicone derivatives, aloe vera, and sorbitol. Natural oil, wax fatty alcohol, hydrocarbon, and a natural plant extract fall within the scope of the emulsifier. Tetrasodium EDTA may be used as a hard water softener.

The soap composition according to the present invention may further comprise an additive such as a bactericide, a dispersant, an antifoaming agent, a solvent, an anti-slime agent, an anti-corrosive agent, a perfume, a dye, a chelating agent, an antioxidant, and antiseptic.

In accordance with still a further embodiment of the present invention, the composition of the present invention is provided as a pharmaceutical composition.

The pharmaceutical composition according to the present invention may comprise a pharmaceutically acceptable carrier or excipient in addition to the active ingredient and may be prepared in a formulation for topical use, such as creams, lotions, ointments (semisolid medicine for external use), microemulsions, gels, pastes, transdermal therapeutic systems (TTS) (e.g., patches, bandages, and the like).

As used herein, the term “pharmaceutically acceptable” means being of such a low toxicity that the subject can endure the effects, without interfering with the activity of the active ingredient used together therewith.

Examples of the pharmaceutically acceptable carrier include lactose, glucose, sucrose, starch (e.g., corn starch, potato starch), cellulose and its derivatives (e.g., sodium carboxymethyl cellulose, ethyl cellulose, etc.), malt, gelatin, talc, solid lubricants (e.g., stearic acid, magnesium stearate, etc.), calcium sulfate, vegetable oils (e.g., peanut oil, cotton seed oil, sesame oil, olive oil, etc.), polyol (e.g., propylene glycol, glycerin, etc.), alginic acid, emulsifiers (e.g., TWEENS), a wetting agent (e.g., sodium lauryl sulfate), colorants, a flavoring agent, a stabilizer, an antioxidant, a preservative, water, saline, and phosphate buffered saline. They are used alone or in combination according to the formulations of the pharmaceutical composition of the present invention.

Excipients also may be selected according to the formulations of the pharmaceutical composition of the present invention. A suspending agent or a dispersant such as sodium carboxymethyl cellulose, methyl cellulose, hydropropylmethyl cellulose, sodium alginate, or polyvinyl pyrrolidone, may be used.

The pharmaceutical composition of the present invention may be administered via an oral or a parenteral route, preferably topically.

The pharmaceutical composition of the present invention may be administered at a daily dose of from 0.001 to 150 mg/kg weight. The composition may be administered in a single dose or it may be spread out over multiple doses per day. For purposes of the present invention, however, the therapeutically effective dose for a certain patient is dependent on various factors including route of administration, patient's age, sex, weight, and general health state. Thus, the dose should not limit the scope of the present invention in any aspects.

In yet another embodiment of the present invention, the composition of the present invention is provided as a food composition.

The food composition of the present invention may be prepared as a health aid food, a special nutrient supplementing food, or a functional beverage.

In addition to the active ingredient, the food composition of the present invention may comprise a sweetener, a flavoring agent, a physiological active ingredient, or mineral.

A sweetener may be used in such an amount as to make food sweet and may be natural or synthetic. Preferable is a natural sweetener. Examples of a natural sweetener include corn syrup, honey, sucrose, fructose, lactose and maltose.

A flavoring agent improves the taste and flavor of food. It may be natural or synthetic. Preferable is a natural flavoring agent. A flavoring agent of natural origin aims to supplement nutrients as well flavoring the food. A natural flavoring agent can be obtained from apples, lemons, citrus, grapes, strawberries, peaches, green tea leaves, Solomon's seal, bamboo leaves, cinnamon, chrysanthemum leaves, jasmine, etc. It is also obtainable from ginseng (red ginseng), bamboo shoots, aloe vera, ginkgo nuts, etc. The natural flavoring agent may be a concentrate in a liquid phase or an extract in a solid phase. A synthetic flavoring agent may also be used. Ester, alcohol, aldehyde, and terpene are known as synthetic flavoring agents.

Examples of the physiological active ingredient include catechins, such as catechin, epicatechin, gallocatechin, and epigallocatechin, and vitamins, such as retinol, ascorbic acid, tocopherol, calciferol, thiamine, and riboflavin.

Among the minerals are calcium, magnesium, chromium, cobalt, copper, fluoride, germanium, iodine, iron, lithium, magnesium, manganese, molybdenum, phosphorus, potassium, selenium, silicon, sodium, sulfur, vanadium, and zinc.

If necessary, the food composition of the present invention may comprise a preservative, an emulsifier, an acidulant, and/or a thickener. It is preferable that the preservative and the emulsifier be used in as low an amount as possible provided that it achieves the goal of addition. Numerically, the amount is in the order of 0.0005 to 0.5% by weight based on the total weight of the food composition.

Examples of the preservatives available in the food composition of the present invention include sodium calcium sorbate, sodium sorbate, potassium sorbate, calcium benzoate, sodium benzoate, potassium benzoate, and EDTA (ethylene diamine tetraacetic acid).

Examples of the emulsifiers available in the food composition of the present invention include acacia gum, carboxymethyl cellulose, xanthan gum, and pectin.

Among the acidulants available for use in the food composition of the present invention are malic acid, fumaric acid, adipic acid, phosphoric acid, gluconic acid, tartaric acid, ascorbic acid, acetic acid, phosphoric acid, etc. The acidulant may be used not only to enhance the taste of the food composition, but also to give the food composition enough acidity to suppress the proliferation of microorganisms.

The thickeners available for use in the food composition of the present invention may be exemplified by a suspending agent, a cohesive agent, a gelling agent and a swelling agent.

In accordance with another aspect thereof, the present invention pertains to a method for treating hair loss.

The method for treating hair loss according to the present invention comprises contacting the composition on the scalp or hair.

As stated above, the composition of the present invention, characterized by the employment of Indian strawberry extracts, exhibits excellent effects in preventing hair loss and promoting hair regrowth, without exhibiting any side effect.

The composition of the present invention may be formulated into hair tonics, shampoos, soaps, foods, and drugs.

A better understanding of the present invention may be obtained through the following examples which are set forth to illustrate the invention, but are not to be construed as limiting the present invention.

### EXAMPLES

### Preparation of Indian Strawberry Extract and Red Ginseng Extract

### Example 1

### Preparation of Indian Strawberry Extract

1-1: Preparation of Indian Strawberry Extract

To 5 liters of water was added 1 kg of dried Indian strawberry herba, followed by heating at 70˜90° C. for about 6 hrs. Filtration through a 2 μm filter afforded an Indian strawberry extract. The resulting liquid extract was used in experiments.

1-2: Preparation of Indian Strawberry Extract

1 kg of dried Indian strawberry herba was immersed at room temperature for 24 hrs in 2 L of 70% ethanol. The filtration of the solution through 2 μm filter afforded an Indian strawberry extract. The resulting liquid extract was used in experiments.

### Example 2

### Preparation of Red Ginseng Extract

2-1: Preparation of Red Ginseng Extract

To 5 liters of water was added 1 kg of finely sliced red ginseng, followed by heating at 70˜90° C. for about 6 hrs. Filtration through a 2 μm filter afforded a red ginseng extract. The resulting liquid extract was used in experiments.

2-2: Preparation of Red Ginseng Extract

1 kg of finely sliced red ginseng was immersed at room temperature for 24 hrs in 2 L of 70% ethanol. The filtration of the solution through 2 μm filter afforded a red ginseng extract. The resulting liquid extract was used in experiments.

### Example 3

### Preparation of Zanthoxylum Planispinum Leaf Extract

3-1: Preparation of Zanthoxylum Planispinum Leaf Extract

To 5 liters of water was added 1 kg of finely sliced zanthoxylum planispinum leaves, followed by heating at 70˜90° C. for about 6 hrs. Filtration through a 2 μm filter afforded a zanthoxylum planispinum leaf extract. The resulting liquid extract was used in experiments.

3-2: Preparation of Zanthoxylum Planispinum Leaf Extract

1 kg of zanthoxylum planispinum leaf aputation is immersed in 2 l of 70% ethanol during 24 hours at room temperature to obtain a liquid extract which is filtered by 2 μm filter paper and zanthoxylum planispinum leaf extract is prepared. The liquid extract itself is used in experiment.

1 kg of finely sliced zanthoxylum planispinum leaves was immersed at room temperature for 24 hrs in 2 L of 70% ethanol. The filtration of the solution through 2 μm filter afforded a zanthoxylum planispinum leaf extract. The resulting liquid extract was used in experiments.

### EXPERIMENTAL EXAMPLES

### Assay for Activity of Preventing Hair Loss and/or Promoting Hair Regrowth

### Experimental Example 1

### Inhibitory Activity Against 5α-Reductase

The extracts obtained in the Preparation Examples were assayed for inhibitory activity against 5 α-reductase as follows.

For use in experiments, 5 α-reductase was obtained from the livers of 8-week-old Sprague-Dawley rats and quantitatively analyzed using a Bradford assay. A protein suspension containing 5 α-reductase at a predetermined level was prepared.

To a mixture of 30 μl of the 5 α-reductase suspension and 60 μl (50 mM Na₂HPO₄ (pH 6.8), 25 mM KCl, 500 mM NADPH) of an enzyme reaction solution was added 10 μl of each of the extracts prepared in the Preparation Examples (Indian strawberry extract, red ginseng extract, Zanthoxylum planispinum leaf extract) or mixtures thereof at various weight ratios to form a final volume of 100 μl.

Radio-labeled testosterone (₃H-Testosterone) was used as a substrate of 5 α-reductase.

The reaction solution was incubated at 37° C. for 20 min to perform the enzymatic reaction. Following the removal of the reaction solvent by evaporation, the residue was dissolved in 20 μl of chloroform, and the solution was loaded on a TLC plate and developed for 30 min with a developing solvent (toluene:acetone=4:1) in a TLC chamber. The developed plate was exposed for three days to Hyperfilm, followed by quantitative analysis with a densitometer.

% Inhibitory activity against 5 α-reductase was evaluated using the following formula, and the results are summarized in Table 1, below.

% Inhibitory activity against 5α-reductase=100(A−B/A)

A: Rate of conversion from testosterone to dihydrotestosterone in the absence of a sample

B: Rate of conversion from testosterone to dihydrotestosterone in the presence of a sample

TABLE 1
% Inhibitory Activity against 5 α-Reductase

| | Inhibitory |
|---|---|
| Sample | Activity (%) |
| Ex. 1-1 | 64.3 |
| Ex. 1-2 | 58.7 |
| Ex. 2-1 | 21.3 |
| Ex. 2-2 | 17.5 |
| Ex. 3-1 | 3.2 |
| Ex. 3-2 | 5.3 |
| A mixture of 9:1 Ex. 1-1:Ex. 2-1 | 77.8 |
| A mixture of 8:2 Ex. 1-1:Ex. 2-1 | 83.5 |
| A mixture of 7:3 Ex. 1-1:Ex. 2-1 | 75.3 |
| A mixture of 6:4 Ex. 1-1:Ex. 2-1 | 65.2 |
| A mixture of 5:5 Ex. 1-1:Ex. 2-1 | 54.3 |
| A mixture of 4:6 Ex. 1-1:Ex. 2-1 | 43.5 |
| A mixture of 3:7 Ex. 1-1:Ex. 2-1 | 34.4 |
| A mixture of 2:8 Ex. 1-1:Ex. 2-1 | 29.8 |
| A mixture of 1:9 Ex. 1-1:Ex. 2-1 | 24.5 |
| A mixture of 9:1 Ex. 1-1:Ex. 3-1 | 74.3 |
| A mixture of 8:2 Ex. 1-1:Ex. 3-1 | 69.3 |
| A mixture of 7:3 Ex. 1-1:Ex. 3-1 | 65.5 |
| A mixture of 6:4 Ex. 1-1:Ex. 3-1 | 56.6 |
| A mixture of 5:5 Ex. 1-1:Ex. 3-1 | 47.1 |
| A mixture of 4:6 Ex. 1-1:Ex. 3-1 | 32.3 |
| A mixture of 3:7 Ex. 1-1:Ex. 3-1 | 21.4 |
| A mixture of 2:8 Ex. 1-1:Ex. 3-1 | 14.5 |
| A mixture of 1:9 Ex. 1-1:Ex. 3-1 | 8.7 |

As is apparent from the data in Table 1, all of the Indian strawberry extract, the red ginseng extract and the zanthoxylum planispinum leaf extract inhibited 5α-reductase, showed the superiority of the Indian strawberry extract to both the red ginseng extract and the zanthoxylum planispinum leaf extract.

As a rule, higher inhibitory activity against 5α-reductase was detected when the extracts were used in combination than alone. In particular, mixtures of the Indian strawberry extract and the red ginseng extract at a weight ratio of from 9:1 to 7:3 were observed to peak in the inhibitory activity.

### Experimental Example 2

### Assay of the Indian Strawberry Extract and/or the Red Ginseng Extract for Activity of Promoting Hair Regrowth in Animal Models

The back of each of C57BL/6 7˜8 week old male mice was carefully shaved in an area of 3˜4 cm² with razor. 24 Hours after shaving, the areas were confirmed to retain no hair. The mice were divided to groups of 7.

A sample (the Indian strawberry extract, the red ginseng extract, the Zanthoxylum planispinum leaf extract, mixtures thereof at various weight ratios) was applied in a proper amount twice a day for two weeks to the shaved back which was then photographed by a digital camera. The images were analyzed with an image analyzer (Image-Pro Plus, U.S.A.). Hair regrowth efficiency was evaluated by the percentage of the area where hair grew newly to the shaved area.

For a control, distilled water was used.

The results are expressed as a mean value±standard deviation in Table 2, below.

TABLE 2
Hair Regrowth Efficiency in Animal Model

| | After | After |
|---|---|---|
| Sample | 1 Week | 2 Weeks |
| Control | 3.97 ± 2.79 | 36.58 ± 11.42 |
| Example 1-1 | 17.74 ± 4.31 | 64.43 ± 8.80 |
| Example 1-2 | 15.22 ± 5.61 | 59.28 ± 5.69 |
| Example 2-1 | 5.34 ± 3.82 | 42.42 ± 5.63 |
| Example 2-2 | 4.52 ± 2.95 | 40.57 ± 4.62 |
| Example 3-1 | 4.27 ± 2.64 | 38.52 ± 8.92 |
| Example 3-2 | 4.36 ± 2.17 | 41.83 ± 8.84 |
| A mixture of 9:1 Ex. 1-1:Ex. 2-1 | 22.15 ± 4.72 | 83.29 ± 7.47 |
| A mixture of 8:2 Ex. 1-1:Ex. 2-1 | 25.57 ± 5.32 | 92.37 ± 8.27 |
| A mixture of 7:3 Ex. 1-1:Ex. 2-1 | 21.62 ± 3.91 | 81.26 ± 5.82 |
| A mixture of 6:4 Ex. 1-1:Ex. 2-1 | 16.73 ± 4.76 | 65.43 ± 6.93 |
| A mixture of 5:5 Ex. 1-1:Ex. 2-1 | 12.31 ± 3.83 | 59.04 ± 9.27 |
| A mixture of 4:6 Ex. 1-1:Ex. 2-1 | 9.21 ± 4.43 | 52.92 ± 7.29 |
| A mixture of 3:7 Ex. 1-1:Ex. 2-1 | 6.47 ± 3.75 | 47.89 ± 8.26 |
| A mixture of 2:8 Ex. 1-1:Ex. 2-1 | 5.82 ± 1.27 | 44.63 ± 10.24 |
| A mixture of 1:9 Ex. 1-1:Ex. 2-1 | 5.57 ± 3.53 | 43.72 ± 11.93 |
| A mixture of 9:1 Ex. 1-1:Ex. 3-1 | 23.15 ± 5.29 | 79.32 ± 7.64 |
| A mixture of 8:2 Ex. 1-1:Ex. 3-1 | 20.14 ± 4.72 | 71.48 ± 8.26 |
| A mixture of 7:3 Ex. 1-1:Ex. 3-1 | 18.62 ± 4.83 | 64.39 ± 6.48 |
| A mixture of 6:4 Ex. 1-1:Ex. 3-1 | 14.75 ± 3.94 | 56.91 ± 5.38 |
| A mixture of 5:5 Ex. 1-1:Ex. 3-1 | 11.92 ± 2.83 | 49.05 ± 8.23 |
| A mixture of 4:6 Ex. 1-1:Ex. 3-1 | 8.85 ± 3.17 | 46.36 ± 7.26 |
| A mixture of 3:7 Ex. 1-1:Ex. 3-1 | 7.27 ± 2.91 | 44.34 ± 4.92 |
| A mixture of 2:8 Ex. 1-1:Ex. 3-1 | 5.75 ± 2.14 | 42.81 ± 6.32 |
| A mixture of 1:9 Ex. 1-1:Ex. 3-1 | 4.64 ± 2.52 | 41.37 ± 5.94 |

On the whole, the data of Table 2 are incident with that of Table 1. The Indian strawberry extract showed remarkably high hair regrowth efficiency compared to both the red ginseng extract and the zanthoxylum planispinum leaf extract. Higher hair regrowth efficiency was detected when the extracts were used in combination than alone. Particularly, mixtures of the Indian strawberry extract and the red ginseng extract at a weight ratio of from 9:1 to 7:3 were observed to peak in hair regrowth efficiency.

### Experimental Example 3

### Clinical Assay of the Indian Strawberry Extract and/or Red Ginseng Extract for Activity of Preventing Hair Loss and Promoting Hair Regrowth

A clinical assay was performed to examine the effect of preventing hair loss and promoting hair regrowth of the extracts of the examples.

Hair tonic compositions were prepared from the Indian strawberry extract, the red ginseng extract and the zanthoxylum planispinum leaf extract, as disclosed in Table 3, below. For comparison, Moxidil® (Hanmi Pharmaceutical, Korea) was used as a positive control.

TABLE 3
Hair Tonic Compositions

| Hair | |
|---|---|
| Tonic No. | Ingredient and Amount (weight %) |
| 1 | Indian strawberry extract of Ex.1-1 (100%) |
| 2 | Red ginseng extract of Ex. 2-1 (100%) |
| 3 | Zanthoxylum planispinum leaf extract of Ex. 3-1 |
| | (100%) |
| 4 | A mixture of 8:2 Ex. 1-1:Ex. 2-1 |
| 5 | A mixture of 9:1 Ex. 1-1:Ex. 3-1 |

150 adult men at ages from 30 to 55 suffering from hair loss were randomly selected and divided into five groups of 30. After washing their hair, they applied the correct amount of each hair tonic to areas of their head experiencing hair loss once or twice a day for three months.

They observed the areas of hair loss with their naked eyes and evaluated the samples for the activity of hair regrowth according to the criterion set below. The results are summarized as mean values in Table 4, below.

TABLE 4
Clinical Test Results of Hair Tonic Compositions

| Hair Tonic No. | Scores of Sensory Evaluation |
|---|---|
| 1 | 3.78 |
| 2 | 2.31 |
| 3 | 2.15 |
| 4 | 4.46 |
| 5 | 4.26 |
| Positive control | 4.21 |

* Evaluation Criteria
5 points: Hair loss was remarkably reduced, and hair grew
4 points: Hair loss was clearly reduced.
3 points: Hair loss was moderately reduced.
2 points: No change in hair loss occurred.
1 Point: Hair loss deteriorated.

As seen in Table 4, the Indian strawberry extract promoted hair regrowth at higher efficiency than did the red ginseng extract or the zanthoxylum planispinum leaf extract. On the whole, higher hair regrowth efficiency was detected when the extracts were used in combination than alone.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method for treating hair loss, comprising applying a composition to a scalp or hair, the composition comprising an Indian strawberry extract as active ingredient.

2. The method according to claim 1, wherein the composition further comprises a red ginseng extract or a red ginseng powder.

3. The method according to claim 1, wherein the composition comprises a mixture of the Indian strawberry extract and a red ginseng extract at a weight ratio of 1:9 to 3:7.

4. The method according to claim 1, wherein the composition further comprises a zanthoxylum planispinum leaf extract.

5. The method according to claim 1, wherein the composition is a hair tonic formulation.

6. The method of claim 1, wherein the composition is a shampoo formulation.

7. The method of claim 1, wherein the composition is a soap formulation.

8. The method of claim 1, wherein the composition is a pharmaceutical formulation.

9. The method of claim 1, wherein the composition is a food formulation.
